(19) United States
(12) Patent Application Publication
(10) Pub. No.: US 2024/0065977 A1
(43) Pub. Date: Feb. 29, 2024

(54) MYDRIATIC AND ANTI-MUSCARINIC AGENT COMPOSITION FOR OPHTHALMIC USE

(71) Applicant: SENTISS PHARMA PRIVATE LIMITED, New Delhi (IN)

(72) Inventors: Rajesh RAO, Gurugram (IN); Vasundera SINGH, SR., Gurugram (IN); Tarun Kumar MANDAL, Gurugram (IN); Deepak BAHRI, Gurugram (IN)

(73) Assignee: SENTISS PHARMA PRIVATE LIMITED, New Delhi (IN)

(21) Appl. No.: 18/270,389

(22) PCT Filed: Dec. 29, 2021
(86) PCT No.: PCT/IB2021/062424

§ 371 Date: Jun. 29, 2023

(30) Foreign Application Priority Data
Dec. 30, 2020 (IN) 202011057315

(51) Int. Cl. A61K 9/08; A61K 31/137; A61K 31/46; A61K 47/02; A61K 47/12; A61K 47/18; A61K 47/36

(52) U.S. Cl. CPC A61K 9/08; A61K 31/137; A61K 31/46; A61K 47/02; A61K 47/12; A61K 47/183; A61K 47/186; A61K 47/36

(57) ABSTRACT

The present invention provides an ophthalmic composition comprising a combination of mydriatic agent and anti-muscarinic agent along with pharmaceutically acceptable excipients, wherein the pH of the composition is in the range of 3.5 to 8.5. The ophthalmic composition of the present invention has synergistic mydriasis effect to induce pupil dilation (Mydriasis) in ophthalmic surgery for pre-operative preparation.

### FIELD OF THE INVENTION

The present invention provides an ophthalmic composition comprising a combination of mydriatic agent and anti-muscarinic agent along with pharmaceutically acceptable excipients, wherein the composition has pH in the range of 3.5 to 8.5. The ophthalmic composition of the present invention has synergistic mydriasis effect to induce pupil dilation (Mydriasis) in ophthalmic surgery for pre-operative preparation.

### BACKGROUND OF THE INVENTION

The present invention relates to an ophthalmic composition comprising a combination of mydriatic agent and anti-muscarinic agent along with the pharmaceutically acceptable excipients.

Phenylephrine and its salts alone is recommended as a vasoconstrictor, decongestant and mydriatic agent for a wide variety of ophthalmic conditions and procedures. A mydriatic is an agent which induces dilation of the pupil. Some of its uses are for pupillary dilation in uveitis, for many surgical procedures and for refraction without cycloplegia. It may also be used for fundoscopy and other diagnostic procedures such as provocative test in patients with narrow profile of anterior chamber angle, differential diagnostics and pupil dilation in ophthalmic surgery for preoperative preparation.

Phenylephrine Hydrochloride is an alpha-1 adrenergic receptor agonist indicated to dilate the pupil. Phenylephrine Hydrochloride acts on the iris dilator muscle. Marketed formulations for Phenylphrine Hydrochloride are available in a concentration of 2.5%, 5% and 10%.

Atropine and its salts act as an anti-muscarinic agent indicated for mydriasis, cycloplegia and penalization of the healthy eye in the treatment of amblyopia. Atropine blocks contraction of the pupillary sphincter muscle. It is indicated in individuals from three months of age or greater, for intended maximal dilation time. Marketed formulations of atropine are available in concentration of 0.5%, 1.0%.

There are evidences that show enhanced mydriatic effect of two drugs of different mechanism. One similar study has shown that 1% Tropicamide, with its parasympathetic antagonistic mechanism of action, was more effective at inducing pupillary dilation than 2.5% phenylephrine, and the combination of 1% Tropicamide and 2.5% Phenylephrine was more effective than multiple drops of single eye drops (Ref: The Comparison of Mydriatic Effect Between Two Drugs of Different Mechanism, Ji-Hyun Park, Young-Chun Lee, and Se-Youp Lee). Further, Mydrimax eye drops (Phenylephrine hydrochloride—5%, Tropicamide—0.8%) is available in market and it is used as a mydriatic remedy for dilation of the pupil for ophthalmic diagnostic and surgical interventions.

Phenylephrine Hydrochloride (5%) and Homatropine Hydrobromide (1%) is available in market under brand name SUNEPHRINE H (Mfg by: Sunways India Pvt Ltd), which acts as a Mydriatic and Cycloplegic. SUNEPHRINE H is indicated for pupil dilation and for pre- and post-operative states when mydriasis and cycloplegia is required. However, this medication has some known side effects which can be extreme or intense, depending upon the clinical condition of the patient. These reactions could conceivably happen dependably and some of them are uncommon however serious.

Atropine has a slow onset of action (40 min) and the pupillary dilatation is accompanied by cycloplegia. Atropine is potent and has the longest duration of action (7 days or more). Systemic side effects of atropine include tachycardia, decreased gastrointestinal motility, and reduced tear production. Below are the comparative mydriatic effects for Atropine, Tropicamide & Homatropine (Ref: https://optometrysmeeting.org/documents/handouts/2019/P450.pdf).

| | Mydriasis | | | |
|---|---|---|---|---|
| Drug | Concentration | Peak | Recovery | |
| Atropine | 0.5%-3% | 30-40 min | 7-12 | days |
| Tropicamide | 0.5%-1% | 20-40 min | 6 | hr |
| Homatropine | 2%-5% | 40-60 min | 1-3 | days |

Concomitant use of Phenylephrine and Atropine (for available marketed formulations) may enhance the pressor effects and induce tachycardia in some patients. Phenylephrine may potentiate the cardiovascular depressant effects of some inhalation anaesthetic agents (Ref: https://www.accessdata.fda.gov/drugsatfda_docs/label/2015/207926s000lbl.pdf).

However, use of Phenylephrine Hydrochloride (2.5%) along with ultra-low concentration of Atropine Sulphate (0.01%) can have synergistic clinical effect with lower side effects. Thus, there is a major need for an ophthalmic composition capable of exhibiting an effective/better mydriasis effect, with lesser side effects and easy to manufacture.

Therefore, the objective of the present invention is to meet this need and to overcome the drawbacks of the prior art by manufacture the ophthalmic composition comprising a combination of mydriatic agent and anti-muscarinic agent in ultra-low concentration along with pharmaceutically acceptable excipients to achieve a better mydriasis effect than the formulations currently available in market.

### OBJECTS OF THE INVENTION

The main object of the present invention is to provide an ophthalmic composition which comprises a mydriatic agent, an anti-muscarinic agent, optionally with one or more pharmaceutically acceptable excipients.

Another object of the present invention is to provide an ophthalmic composition which is useful for pupil dilation in ophthalmic surgery for pre-operative preparation.

### SUMMARY OF THE INVENTION

The present invention provides an ophthalmic composition comprising a combination of mydriatic agent and anti-muscarinic agent along with pharmaceutically acceptable excipients, wherein the composition has pH in the range of 3.5 to 8.5.

The present invention further provides an ophthalmic composition which has synergistic mydriasis effect to induce pupil dilation in ophthalmic surgery for pre-operative preparation.

The present invention furthermore provides an ophthalmic solution for dilation of pupil, characterized by installing a combination of mydriatic agent and anti-muscarinic agent along with pharmaceutically acceptable excipients to the patient in need thereof.

In another embodiment, also disclosed is a method of preparing an ophthalmic composition comprising a combination of mydriatic agent and anti-muscarinic agent along with pharmaceutically acceptable excipients, to achieve better mydriasis effect to induce pupil dilation in ophthalmic surgery for pre-operative preparation.

In one embodiment, the ophthalmic composition of the present invention can be administered as a solution, suspension, ointment, gel and emulsion in a suitable ophthalmic vehicle or pharmaceutically acceptable excipients.

### DETAILED DESCRIPTION OF THE INVENTION

The present invention provides an ophthalmic composition comprising a combination of mydriatic agent and anti-muscarinic agent along with pharmaceutically acceptable excipients, wherein the composition has pH in the range of 3.5 to 8.5.

The ophthalmic composition of the present invention has synergistic mydriasis effect to induce pupil dilation in ophthalmic surgery for pre-operative preparation.

The present invention provides an ophthalmic solution for dilation of pupil, characterized by installing a combination of mydriatic agent and anti-muscarinic agent along with pharmaceutically acceptable excipients.

Preferred mydriatic agents for use in the ophthalmic composition of the present invention, to dilate the pupil during surgery, include, but not limited to, alpha-1 adrenergic receptor agonists. The alpha-1 adrenergics will be preferred because they provide mydriasis effect but not cycloplegia. Alpha-1 adrenergics are thus shorter acting, causing mydriasis during a surgical procedure and allowing the pupil to return to its normal state shortly after completion of the procedure. Examples of suitable adrenergic receptor agonists/mydriatic agents include, but not limited to, phenylephrine, epinephrine, oxymetazoline, its derivatives, salts thereof. Other agents that cause mydriasis, and particularly short-acting mydriatic agents, are also intended to be encompassed by the present invention. The mydriatic agent is present in a concentration of about 0.1% w/v to 10% w/v of the composition. Preferably, in an amount of about 2.5% w/v of the composition.

Examples of suitable anti-muscarinic agent include, but are not limited to, Atropine, Homatropine, Scopolamine, its derivatives, salts thereof, wherein the anti-muscarinic agent is present in a concentration of about 0.001% w/v to about 1% w/v.

However, the anti-muscarinic agents may cause side effects of blurred vision and photophobia. These side effects may be overcome by administering ultra-low concentration about 0.001% w/v to about 0.1% w/v of anti-muscarinic agents, in combination with one or more mydriatic agent, to achieve the desired therapeutic effect.

The synergistic effect of an ophthalmic composition of present invention comprising a combination of a mydriatic agent (phenylephrine) and anti-muscarinic agent (atropine) may afford effective dilation of pupil during ophthalmic surgery for pre-operative preparation wherein one or even all of the ultra-low concentration of anti-muscarinic agents would not be sufficient to have a therapeutic effect when the respective anti-muscarinic agent is used in monotherapy.

The ophthalmic composition to be administered according to the methods of some embodiments provided herein can be readily formulated with, prepared with, or administered with, the pharmaceutically acceptable excipients.

In one embodiment, the ophthalmic composition of the present invention can be administered as a solution, suspension, ointment, gel and emulsion in a suitable ophthalmic vehicle or pharmaceutically acceptable excipients.

In one embodiment, the ophthalmic composition of the present invention comprises mydriatic agent and anti-muscarinic agent in dissolved form.

In another preferred embodiment, the pharmaceutical composition of the present invention comprising phenylephrine and atropine may show effective dilation (Mydriasis) and simultaneously prohibits paralysis of the ciliary muscles of the eye (Cycloplegia) due to the ultralow concentration of Atropine.

In another embodiment, the pharmaceutical composition of the present invention provides methods of dilating the pupil through administering a composition comprising a combination of phenylephrine hydrochloride and atropine sulphate topically to a patient in need thereof, wherein the dilation is achieved during ophthalmic surgery for pre-operative preparation.

In yet another embodiment, the pharmaceutical composition of the present invention provides methods of performing certain ocular testing such as ultrasonography, provocative closed angle glaucoma test, retinoscopy, compromised circulation (i.e., blanching test), refraction, fundus examination by administering a composition comprising a combination of phenylephrine hydrochloride and atropine sulphate topically to a patient in need thereof, along with pharmaceutically acceptable excipients.

In another embodiment, the present invention provides methods of aiding surgical procedures requiring visualization of the posterior chamber comprising administering a composition comprising a combination of phenylephrine hydrochloride and atropine sulphate.

The composition of present invention in addition to active ingredients may also contain pharmaceutical acceptable excipients, such as anti-oxidants, polymers, tonicity adjusting agents, chelating agents, dispersing agents, polymers, emulsifiers, humectants, thickening agents, oils, surfactants, co-surfactants, buffering agents, preservatives and solvents etc.

In yet another embodiment, the preferred preservatives include, but are not limited to, benzalkonium chloride, benzethonium chloride, chlorhexidine gluconate, benzododecinium bromide, methylbenzethonium chloride, cetalkonium chloride, cetylpyridinium chloride, cetrimonium, cetrimide, dofanium chloride, tetraethylammonium bromide, didecyldimethylammonium chloride, domiphen bromide, polyquaternium-1 (Polyquad®), stabilized oxychloro complex, 1-phenyl ethanol, phenyl propanol, phenyl mercuric acetate, phenyl mercuric nitrate, phenyl mercuric borate, chlorhexidine acetate or gluconate, chlorocresol, benzoic acid, benzyl alcohol, butylparaben, propylparaben, methylparaben, chlorobutanol, Sorbic Acid, phenoxyethanol, sodium methyl paraben, sodiumpropyl paraben, thimerosal, and mixtures thereof in an effective amount of preferably from 0.005% to 0.5% (w/v).

In another embodiment of the present invention, the chelating agent is selected from a group include, but not limited to, edetate disodium (EDTA), edetate disodium dihydrate, trisodium phosphate, diammonium citrate, galactaric acid, galacturonic acid, gluconic acid, glucuronic acid, cyclodextrin, potassium citrate, the potassium salt of ethylenediamine-tetra (methylene phosphonic acid) (“EDTMP”), sodium citrate, sodium citrate dihydrate, sodium EDTMP and/or combinations/mixtures thereof.

In another preferred embodiment of the present invention, the polymers include, but are not limited to, methylcellulose, hydroxypropyl methylcellulose (hypromellose), poloxamers, carboxymethyl cellulose, hydroxyethyl cellulose, polyvinyl alcohol, povidone, polyethylene glycol, hyaluronic acid (Sodium Hyaluronate), polygalacturonic acid, xyloglucan, carbopol, polycarbophil, gellan gum, and/or mixtures thereof.

In another embodiment, the composition of the present invention includes anti-oxidant that include, but are not limited to, sodium metabisulfite, potassium, citric acid, tartaric acid, ascorbic acid, phosphoric acid, sodium bisulfite and/or mixtures thereof.

In another embodiment, the composition of the present invention may include one or more buffering agents which include, but are not limited to, acetate buffers, citrate buffers, phosphate buffers, hydrochloric acid, sodium hydroxide, sodium dihydrogen phosphate dihydrate, dibasic sodium phosphate heptahydrate, monobasic sodium phosphate, sodium citrate dihydrate, citric acid, citric acid monohydrate, ε-aminocaproic acid, triethanolamine and/or mixtures thereof.

In yet preferred embodiment of the present invention, tonicity adjusting agents may be added that include, without limitation, glycerin, sorbitol, sodium chloride, potassium chloride, mannitol, dextrose, and propylene glycol, sodium borate or boric acid and/or mixtures thereof.

In another embodiment of the present invention, the pH adjusting agent include, but are not limited to, hydrochloric acid, citric acid, phosphoric acid, acetic acid, tartaric acid, sodium hydroxide, potassium hydroxide, sodium carbonate and sodium hydrogen carbonate and/or mixtures thereof.

In another embodiment of the present invention, suitable vehicle is water, saline, phosphate buffered saline, and/or mixtures thereof.

In another embodiment, the composition of the present invention does not contain any preservative and thus can be supplied in multi-dose preservative-free containers.

In yet another embodiment, the ophthalmic composition of the present invention, is devoid of any preservative and thus can be supplied in unit-dose container.

In another embodiment, also disclosed is a method of preparing an ophthalmic composition comprising a combination of mydriatic agent and anti-muscarinic agent along with pharmaceutically acceptable excipients, to achieve better mydriasis effect to induce pupil dilation in ophthalmic surgery for pre-operative preparation.

The terms “treatment,” “treat,” “treating,” and the like, are meant to include slowing or reversing the progression of a disease or disorder. These terms also include alleviating, ameliorating, attenuating, eliminating, or reducing one or more symptoms of a disease or disorder or condition, even if the disease or disorder or condition is not actually eliminated and even if progression of the disease or disorder or condition is not itself slowed or reversed.

The main embodiment of the present invention provides a pharmaceutical composition comprising a mydriatic agent, anti-muscarinic agent optionally with one or more pharmaceutically acceptable excipients, wherein the composition has pH in the range of 3.5 to 8.5.

In another embodiment of the present invention, wherein the mydriatic agent is phenylephrine hydrochloride or its pharmaceutically acceptable salts.

In another embodiment of the present invention, wherein the mydriatic agent is present in a concentration of about 0.1% w/v to 10% w/v of the composition.

In another preferred embodiment of the present invention, wherein the mydriatic agent is present in a concentration of about 2.5% w/v of the composition.

In another embodiment of the present invention, wherein the anti-muscarinic agent is atropine sulphate or its pharmaceutically acceptable salts.

In another embodiment of the present invention, wherein the anti-muscarinic agent is present in a concentration of about 0.001% w/v to about 0.1% w/v of the composition.

In another preferred embodiment of the present invention, wherein the anti-muscarinic agent is present in a concentration of about 0.01% w/v of the composition.

In another embodiment of the present invention, wherein the pharmaceutically acceptable excipients are selected from the group comprising preservative, chelating agent, anti-oxidant, polymer, buffering agent, tonicity agent, pH adjusting agent and/or mixtures thereof.

In another embodiment of the present invention, wherein the preservative is selected from the group comprising benzalkonium chloride, benzethonium chloride, chlorhexidine gluconate, benzododecinium bromide, methylbenzethonium chloride, cetalkonium chloride, cetylpyridinium chloride, cetrimonium, cetrimide, dofanium chloride, tetraethylammonium bromide, didecyldimethylammonium chloride, domiphen bromide, polyquaternium-1 (Polyquad®), stabilized oxychloro complex, phenyl ethanol, phenyl propanol, phenyl mercuric acetate, phenyl mercuric nitrate, phenyl mercuric borate, chlorhexidine acetate or gluconate, chlorocresol, benzoic acid, benzyl alcohol, butylparaben, propylparaben, methylparaben, chlorobutanol, Sorbic Acid, phenoxyethanol, sodium methyl paraben, sodiumpropyl paraben, thimerosal, and/or mixtures thereof.

In another embodiment of the present invention, wherein the chelating agent is selected from the group comprising edetate disodium (EDTA), edetate disodium dihydrate, trisodium phosphate, diammonium citrate, galactaric acid, galacturonic acid, gluconic acid, glucuronic acid, cyclodextrin, potassium citrate, the potassium salt of ethylenediamine-tetra (methylene phosphonic acid) (“EDTMP”), sodium citrate, sodium citrate dihydrate, sodium EDTMP and/or mixtures thereof.

In another embodiment of the present invention, wherein the anti-oxidant is selected from the group comprising sodium metabisulfite, potassium metabisulfite, citric acid, tartaric acid, ascorbic acid, phosphoric acid, sodium bisulfite and/or mixtures thereof.

In another embodiment of the present invention, wherein the polymer is selected from the group comprising methylcellulose, hydroxypropyl methylcellulose (hypromellose), poloxamers, carboxymethyl cellulose, hydroxyethyl cellulose, polyvinyl alcohol, povidone, polyethylene glycol, hyaluronic acid (Sodium Hyaluronate), polygalacturonic acid, xyloglucan, carbopol, polycarbophil, gellan gum, and/or mixtures thereof.

In another embodiment of the present invention, wherein the buffering agent is selected from the group comprising acetate buffers, citrate buffers, phosphate buffers, hydrochloric acid, sodium hydroxide, sodium dihydrogen phosphate dihydrate, dibasic sodium phosphate heptahydrate, monobasic sodium phosphate, sodium citrate dihydrate, citric acid, citric acid monohydrate, ε-aminocaproic acid, triethanolamine and/or mixtures thereof.

In another embodiment of the present invention, wherein the tonicity agent is selected from the group comprising glycerin, sorbitol, sodium chloride, potassium chloride, mannitol, dextrose, and propylene glycol, sodium borate or Boric acid and/or mixtures thereof.

In another embodiment of the present invention, wherein the pH adjusting agent is selected from the group comprising hydrochloric acid, citric acid, phosphoric acid, acetic acid, tartaric acid, sodium hydroxide, potassium hydroxide, sodium carbonate and sodium hydrogen carbonate and/or mixtures thereof.

In another embodiment of the present invention, wherein the composition is administered either once a day, twice a day or thrice a day to each eye.

In another embodiment of the present invention, wherein the composition is in the form of solution.

In another embodiment of the present invention, wherein the method of using an ophthalmic composition for pupil dilation comprises topically administering the composition comprising a mydriatic agent, anti-muscarinic agent optionally with one or more pharmaceutically acceptable excipients, wherein the composition has pH in the range of 3.5 to 8.5.

In another embodiment of the present invention, wherein the composition is useful for pupil dilation in ophthalmic surgery for pre-operative preparation.

In another embodiment of the present invention, wherein the composition comprising a combination of phenylephrine or a pharmaceutically acceptable salt thereof in an amount of 2.5% w/v and atropine or a pharmaceutically acceptable salt thereof in an amount of 0.01% w/v, optionally with one or more pharmaceutically acceptable excipients, wherein the combination results in a synergistic effect.

In another embodiment of the present invention, wherein the composition comprising a combination of phenylephrine or its pharmaceutically acceptable salt thereof in an amount of 2.5% w/v and atropine or a pharmaceutically acceptable salt thereof in an amount of 0.01% w/v, optionally with one or more pharmaceutically acceptable excipients, wherein the combination results in reduced side effects.

In another embodiment of the present invention, wherein the composition comprising a combination of phenylephrine or its pharmaceutically acceptable salt thereof in an amount of 2.5% w/v and atropine or a pharmaceutically acceptable salt thereof in an amount of 0.01% w/v, optionally with one or more pharmaceutically acceptable excipients, wherein the combination results in reduced local irritation effect.

Another embodiment of the present invention provides a method of using an ophthalmic composition for pupil dilation comprising topically administering the composition.

In another embodiment of the present invention wherein the composition is useful for pupil dilation in ophthalmic surgery for pre-operative preparation.

The following are the exemplary ophthalmic compositions in accordance with the present invention suitable for ophthalmologic pre-operative procedures.

### Examples

The scope of the invention is illustrated by the following examples as disclosed herein which are not meant to restrict the scope of the invention in any manner whatsoever.

The term ‘q.s.’ wherever appears in the examples is an abbreviation for ‘quantity sufficient’ which is the amount of the excipient in such quantities that is just sufficient for its use in the composition of the present invention.

### Examples 1-3

| | | Example 1 | Example 2 | Example 3 |
|---|---|---|---|---|
| S. No | Ingredients | (mg/mL) | (mg/mL) | (mg/mL) |
| 1. | Phenylephrine | 25.00 | 25.00 | 25.00 |
| | Hydrochloride | | | |
| 2. | Atropine Sulfate | 0.10 | 0.10 | 0.10 |
| 3. | Benzalkonium Chloride | 0.10-1.00 | 0.10-5.00 | 0.10-2.00 |
| 4. | Edetate Disodium | 1.00-5.00 | 0.10-2.50 | 1.00-5.00 |
| 5. | Hydroxy Propyl Methyl | — | — | 5.00-20.00 |
| | Cellulose E-5 | | | |
| 6. | Hydroxy Propyl Methyl | 3.00-20.00 | — | — |
| | Cellulose E-15 | | | |
| 7. | Hypromellose 2910 | — | 5.00-20.00 | — |
| 8. | Sodium MetaBisulfite | 2.00-10.00 | 2.00-10.00 | 2.00-10.00 |
| 9. | Citric Acid | 1.16-10.00 | — | — |
| 10. | Monobasic Sodium | — | 8.00-13.00 | — |
| | Phosphate | | | |
| 11. | Dibasic Sodium Phosphate | — | 3.00-8.00 | — |
| 12. | Sodium Citrate Dihydrate | 7.5-15.00 | — | — |
| 13. | Hydrochloric Acid | q.s. to adjust pH | q.s. to adjust pH | q.s. to adjust pH |
| | | 3.5-8.5 | 3.5-8.5 | 3.5-8.5 |
| 14. | Sodium Hydroxide | q.s. to adjust pH | q.s. to adjust pH | q.s. to adjust pH |
| | | 3.5-8.5 | 3.5-8.5 | 3.5-8.5 |
| 15. | WFI | q.s. to 1 mL | q.s. to 1 mL | q.s. to 1 mL |

### Examples 4 and 5

| | | Example 4 | Example 5 |
|---|---|---|---|
| S. No | Ingredients | (mg/mL) | (mg/mL) |
| 1. | Phenylephrine | 25.00 | 25.00 |
| | Hydrochloride | | |
| 2. | Atropine Sulfate | 0.10 | 0.10 |
| 3. | Benzalkonium Chloride | 0.10-1.00 | 0.10-5.00 |
| 4. | Edetate Disodium | 1.00-5.00 | — |
| 5. | Polyvinyl Alcohol | 14.00-30.00 | — |
| 6. | Povidone | 6.00-10.00 | — |
| 7. | Sodium Hyaluronate | — | 4.00-20.00 |
| 8. | Sodium MetaBisulfite | 2.00-5.00 | 2.00-10.00 |
| 9. | Monobasic Sodium | — | 2.50-10.00 |
| | Phosphate | | |
| 10. | Dibasic Sodium Phosphate | — | 1.00-5.00 |
| 11. | Sodium Chloride | 5.00-8.46 | 5.00-10.00 |
| 12. | Hydrochloric Acid | q.s. to adjust pH | q.s. to adjust pH |
| | | 3.5-8.5 | 3.5-8.5 |
| 13. | Sodium Hydroxide | q.s. to adjust pH | q.s. to adjust pH |
| | | 3.5-8.5 | 3.5-8.5 |
| 14. | WFI | q.s. to 1 mL | q.s. to 1 mL |

### Example 6

| S. No | Ingredients | (mg/mL) |
|---|---|---|
| 1. | Phenylephrine Hydrochloride | 25.00 |
| 2. | Atropine Sulfate | 0.10 |
| 3. | Benzalkonium Chloride | 0.20 |
| 4. | Hydroxy Propyl Methyl Cellulose E-15 | 3.00 |
| 5. | Edetate disodium dihydrate | 1.00 |
| 6. | Citric Acid (Anhydrous) | 1.16 |
| 7. | Sodium Citrate Dihydrate | 7.50 |
| 8. | Sodium Metabisulfite | 2.00 |
| 9. | Water for Injection (WFI) | q.s. to 1 mL |

### Manufacturing Process:

1. Water for injection (WFI) was transferred into a tank and nitrogen gas was purged to bring the dissolved oxygen level below 2 ppm.
2. To water for injection of step 1, hydroxy propyl methyl cellulose was added and dissolved under stirring.
3. Edetate disodium dihydrate was added to solution of step 2 and dissolved under stirring
4. Citric acid anhydrous was added to solution of step 3 and dissolved under stirring.
5. Sodium citrate dihydrate was added to solution of step 4 and dissolved under stirring.
6. Benzalkonium chloride solution (50%) was added to solution of step 5 and dissolved under stirring.
7. Sodium metabisulfite was added to solution of step 6 and dissolved under stirring.
8. Phenylephrine Hydrochloride was added to solution of step 7 and dissolved under stirring.
9. Atropine Sulfate was added to solution of step 8 and dissolved under stirring.
10. The final volume was made up to 100% with WFI to form a bulk solution.
11. The bulk solution was then stirred for 25±5 minutes.
12. pH of the bulk solution was checked (pH limit-4.5-5.5).
13. The bulk solution was then filtered and filled into vials.

### Stability Studies:

The pharmaceutical compositions prepared according to Example 6 were packed in three piece ethylene oxide sterilized, opaque white LDPE vials with ethylene oxide sterilized, opaque white nozzles and ethylene oxide sterilized opaque white HDPE caps and then packed in aqueous coated carton. The packed vials were subjected to accelerated stability studies by storing at storage conditions 40° C./Not more than 25% RH (Upright & Inverted) and long term stability studies by storing at storage conditions of 25° C./40% RH (Upright & Inverted) and 30° C./35% RH (Upright & Inverted) for 3 months. The results of the accelerated and long term stability studies are tabulated in Table 1 and 2 respectively.

TABLE 1
Accelerated Stability Studies of Phenylephrine hydrochloride 2.5%
and Atropine sulphate 0.01% ophthalmic solution
Phenylephrine hydrochloride 2.5% and Atropine sulphate 0.01% ophthalmic
solution (5 mL in 5 mL three piece white opaque LDPE vial
and white opaque HDPE cap)
Condition: 40° C./NMT 25% RH

| Test | | 1 Month | 1 Month | 3 Month | 3 Month |
|---|---|---|---|---|---|
| Parameters | Initial | (Upright) | (Inverted) | (Upright) | (Inverted) |
| Appearance | Clear, | Clear, | Clear, | Clear, | Clear, |
| | Colorless | Colorless | Colorless | Colorless | Colorless |
| | solution | solution | solution | solution | solution |
| pH | 4.89 | 4.82 | 4.81 | 4.72 | 4.71 |
| Osmolality | 359 | 361 | 360 | 365 | 365 |
| (mOsmol/kg) | | | | | |
| Viscosity (cps) | 2.24 | 2.25 | 2.23 | 2.17 | 2.19 |
| Assay of | 98.3 | 100.7 | 100.7 | 101.5 | 101.2 |
| Phenylephrine | | | | | |
| Hydrochloride | | | | | |
| (%) | | | | | |
| Assay of | 101.5 | 100.9 | 101.0 | 100.8 | 100.6 |
| Atropine Sulfate | | | | | |
| (%) | | | | | |
| Content of | 100.2 | 99.5 | 96.5 | 99.3 | 96.8 |
| Benzalkonium | | | | | |
| Chloride (%) | | | | | |

Related Substances
Related Substances of Phenylephrine Hydrochloride

| | | | | | |
|---|---|---|---|---|---|
| Phenylephrine | 0.04 | 0.10 | 0.09 | 0.08 | 0.08 |
| Related | | | | | |
| Compound-C (%) | | | | | |
| Norphenylephrine | 0.01 | 0.04 | 0.04 | 0.04 | 0.04 |
| Hydrochloride | | | | | |
| (%) | | | | | |
| Phenylephrine | ND | ND | ND | ND | ND |
| Related | | | | | |
| Compound-E (%) | | | | | |
| 3-Hydroxy | ND | ND | ND | ND | ND |
| acetophenone (%) | | | | | |
| Naproxen^{#} (%) | NA | ND | ND | ND | ND |
| Specified | NA | 0.10 | 0.10 | 0.09 (RRT-1.70) | 0.09 (RRT-1.70) |
| unidentified | | | | | |
| impurity at RRT | | | | | |
| 1.67 | | | | | |
| Any unspecified | BDL | 0.05 | 0.05 | 0.09 | 0.08 |
| individual | | | | | |
| impurity (%) at | | | | | |
| RRT-0.53 | | | | | |
| Total Impurities | 0.05 | 0.29 | 0.28 | 0.30 | 0.29 |
| (%) | | | | | |

Related substances of Atropine Sulfate

| | | | | | |
|---|---|---|---|---|---|
| Tropic acid (%) | 0.16 | 0.25 | 0.24 | 0.61 | 0.64 |
| Apoatropine (%) | ND | ND | ND | ND | ND |
| Hyoscyamine | NA | ND | ND | ND | ND |
| Related | | | | | |
| Littorine^{#} (%) | NA | 0.01 | 0.01 | ND | ND |
| Any unspecified | ND | ND | ND | ND | ND |
| individual | | | | | |
| impurity (%) | | | | | |
| Total Impurities | 0.16 | 0.25 | 0.24 | 0.61 | 0.64 |
| (%) | | | | | |

TABLE 2
Long Term Stability Studies of Phenylephrine hydrochloride 2.5%
and Atropine sulphate 0.01% ophthalmic solution
Phenylephrine hydrochloride 2.5% and Atropine sulphate 0.01% ophthalmic
solution (5 mL in 5 mL three piece white opaque LDPE vial
and white opaque HDPE cap)

| | | Condition: 25° C./40% RH | | Condition: 30° C./35% RH | |
|---|---|---|---|---|---|
| Test | | 3 Month | 3 Month | 3 Month | 3 Month |
| Parameters | Initial | (Upright) | (Inverted) | (Upright) | (Inverted) |
| Appearance | Clear, | Clear, | Clear, | Clear, | Clear, |
| | Colorless | Colorless | Colorless | Colorless | Colorless |
| | solution | solution | solution | solution | solution |
| pH | 4.89 | 4.82 | 4.81 | 4.78 | 4.77 |
| Osmolality | 359 | 362 | 361 | 364 | 364 |
| (mOsmol/kg) | | | | | |
| Viscosity (cps) | 2.24 | 2.20 | 2.21 | 2.21 | 2.21 |
| Assay of | 98.3 | 100.5 | 100.9 | 100.7 | 100.7 |
| Phenylephrine | | | | | |
| Hydrochloride | | | | | |
| (%) | | | | | |
| Assay of | 101.5 | 100.8 | 100.7 | 100.9 | 100.6 |
| Atropine | | | | | |
| Sulfate (%) | | | | | |
| Content of | 100.2 | 97.4 | 97.2 | 96.8 | 95.5 |
| Benzalkonium | | | | | |
| Chloride (%) | | | | | |

Related Substances
Related Substances of Phenylephrine Hydrochloride

| | | | | | |
|---|---|---|---|---|---|
| Phenylephrine | 0.04 | 0.04 | 0.04 | 0.06 | 0.06 |
| Related | | | | | |
| Compound-C | | | | | |
| (%) | | | | | |
| Norphenylephrine | 0.01 | 0.02 | 0.02 | 0.03 | 0.03 |
| Hydrochloride | | | | | |
| (%) | | | | | |
| Phenylephrine | ND | ND | ND | ND | ND |
| Related | | | | | |
| Compound-E | | | | | |
| (%) | | | | | |
| 3-Hydroxy | ND | ND | ND | ND | ND |
| acetophenone | | | | | |
| Naproxen^{#} (%) | NA | ND | ND | ND | ND |
| Specified | NA | 0.07 (RRT-1.70) | 0.07 (RRT-1.70) | 0.08 (RRT-1.70) | 0.09 (RRT-1.70) |
| unidentified | | | | | |
| impurity at | | | | | |
| RRT 1.67 | | | | | |
| Any unspecified | BDL | BDL | BDL | 0.06 (RRT-0.53) | 0.06 (RRT-0.53) |
| individual | | | | | |
| impurity (%) | | | | | |
| Total Impurities | 0.05 | 0.13 | 0.13 | 0.23 | 0.24 |
| (%) | | | | | |

Related substances of Atropine Sulfate

| | | | | | |
|---|---|---|---|---|---|
| Tropic acid (%) | 0.16 | 0.19 | 0.18 | 0.34 | 0.32 |
| Apoatropine | ND | ND | ND | ND | ND |
| (%) | | | | | |
| Hyoscyamine | NA | ND | ND | ND | ND |
| Related | | | | | |
| Littorine^{#} (%) | NA | ND | ND | ND | ND |
| Any unspecified | ND | ND | ND | ND | ND |
| individual | | | | | |
| impurity (%) | | | | | |
| Total Impurities | 0.16 | 0.19 | 0.18 | 0.34 | 0.32 |
| (%) | | | | | |

ND—Not detected
NMT—Not more than
BQL—Below qualification limit
BDL—below disregard limit
^{#}Process related impurity and same is not considered under total impurities

### CONCLUSION

The results of the stability study indicate that no significant change in appearance, assay of phenylephrine hydrochloride, assay of atropine sulfate, content of benzalkonium chloride, pH, osmolality, viscosity, related substances and total impurities were observed. Hence, it is concluded that the Phenylephrine hydrochloride 2.5% and Atropine sulphate 0.01% ophthalmic solution are stable under accelerated & long term conditions.

### Utility of the Present Invention

The present inventors have provided an ophthalmic composition comprising a combination of mydriatic agent and anti-muscarinic agent along with pharmaceutically acceptable excipients, wherein the pH of the composition is in the range of 3.5 to 8.5. The ophthalmic compositions of the present invention have synergistic mydriasis effect to induce pupil dilation (Mydriasis) in ophthalmic surgery for pre-operative preparation.

1. An ophthalmic composition comprising phenylephrine or its pharmaceutically acceptable salt, atropine or its pharmaceutically acceptable salt, and optionally one or more pharmaceutically acceptable excipients, wherein the composition has pH in the range of 3.5 to 8.5.

2. (canceled)

3. The ophthalmic composition as claimed in claim 1, wherein the phenylephrine or its pharmaceutically acceptable salt is present in a concentration of about 0.1% w/v to 10% w/v of the composition.

4. The ophthalmic composition as claimed in claim 3, wherein the phenylephrine or its pharmaceutically acceptable salt is present in a concentration of about 2.5% w/v of the composition.

5. (canceled)

6. The ophthalmic composition as claimed in claim 1, wherein the atropine or its pharmaceutically acceptable salt is present in a concentration of about 0.001% w/v to about 0.1% w/v of the composition.

7. The ophthalmic composition as claimed in claim 6, wherein the atropine or its pharmaceutically acceptable salt is present in a concentration of about 0.01% w/v of the composition.

8. The ophthalmic composition as claimed in claim 1, wherein the one or more pharmaceutically acceptable excipients are selected from the group consisting of a preservative, a chelating agent, an anti-oxidant, polymer, a buffering agent, a tonicity agent, a pH adjusting agent, and any combinations thereof.

9. The ophthalmic composition as claimed in claim 8, wherein the preservative is selected from the group consisting of benzalkonium chloride, benzethonium chloride, chlorhexidine gluconate, benzododecinium bromide, methylbenzethonium chloride, cetalkonium chloride, cetylpyridinium chloride, cetrimonium, cetrimide, dofanium chloride, tetraethylammonium bromide, didecyldimethylammonium chloride, domiphen bromide, polyquaternium-1 (Polyquad®), stabilized oxychloro complex, phenyl ethanol, phenyl propanol, phenyl mercuric acetate, phenyl mercuric nitrate, phenyl mercuric borate, chlorhexidine acetate or gluconate, chlorocresol, benzoic acid, benzyl alcohol, butyl paraben, propylparaben, methyl paraben, chlorobutanol, sorbic acid, phenoxyethanol, sodium methyl paraben, sodiumpropyl paraben, thimerosal, and/or any combinations thereof.

10. The ophthalmic composition as claimed in claim 8, wherein the chelating agent is selected from the group consisting of edetate disodium (EDTA), edetate disodium dihydrate, trisodium phosphate, diammonium citrate, galactaric acid, galacturonic acid, gluconic acid, glucuronic acid, cyclodextrin, potassium citrate, the potassium salt of ethylenediamine-tetra (methylene phosphonic acid) (“EDTMP”), sodium citrate, sodium citrate dihydrate, sodium EDTMP, and any combinations thereof.

11. The ophthalmic composition as claimed in claim 8, wherein the anti-oxidant is selected from the group consisting of sodium metabisulfite, potassium metabisulfite, citric acid, tartaric acid, ascorbic acid, phosphoric acid, sodium bisulfite, and any combinations thereof.

12. The ophthalmic composition as claimed in claim 8, wherein the polymer is selected from the group consisting of methylcellulose, hydroxypropyl methylcellulose (hypromellose), poloxamers, carboxymethyl cellulose, hydroxyethyl cellulose, polyvinyl alcohol, povidone, polyethylene glycol, hyaluronic acid (sodium hyaluronate), polygalacturonic acid, xyloglucan, carbopol, polycarbophil, gellan gum, and any combinations thereof.

13. The ophthalmic composition as claimed in claim 8, wherein the buffering agent is selected from the group consisting of acetate buffers, citrate buffers, phosphate buffers, hydrochloric acid, sodium hydroxide, sodium dihydrogen phosphate dihydrate, dibasic sodium phosphate heptahydrate, monobasic sodium phosphate, sodium citrate dihydrate, citric acid, citric acid monohydrate, ε-aminocaproic acid, triethanolamine, and any combinations thereof.

14. The ophthalmic composition as claimed in claim 8, wherein the tonicity agent is selected from the group consisting of glycerin, sorbitol, sodium chloride, potassium chloride, mannitol, dextrose, and propylene glycol, sodium borate, boric acid, and any combinations thereof.

15. The ophthalmic composition as claimed in claim 8, wherein the pH adjusting agent is selected from the group consisting of hydrochloric acid, citric acid, phosphoric acid, acetic acid, tartaric acid, sodium hydroxide, potassium hydroxide, sodium carbonate, sodium hydrogencarbonate, and any combinations thereof.

16. (canceled)

17. The ophthalmic composition as claimed in claim 1, wherein the composition is in the form of a solution.

18. The ophthalmic composition of claim 1, comprising a combination of phenylephrine or its pharmaceutically acceptable salt in an amount of 2.5% w/v and atropine or its pharmaceutically acceptable salt in an amount of 0.01% w/v, optionally with one or more pharmaceutically acceptable excipients.

19. (canceled)

20. A method of using an ophthalmic composition for pupil dilation comprising topically administering the composition as claimed in claim 1.

21. The method as claimed in claim 20, wherein the pupil dilation is induced during pre-operative preparation for ophthalmic surgery.

22. The method as claimed in claim 20, wherein the composition is administered either once a day, twice a day or thrice a day to each eye.
